(19) United States
(12) Patent Application Publication
(10) Pub. No.: US 2006/0282088 A1
(43) Pub. Date: Dec. 14, 2006

(54) Apparatus and methods for delivering sutures

(76) Inventor: Timothy J. Ryan, Menlo Park, CA (US)

Correspondence Address: Vista IP Law Group LLP, 2040 MAIN STREET, 9TH FLOOR, IRVINE, CA, 92614, US

(21) Appl. No.: 11/148,814

(22) Filed: Jun. 8, 2005

(51) Int. Cl. A61B 17/04

(52) U.S. Cl. 606/144

(57) ABSTRACT

An apparatus for delivering a suture includes a delivery sheath and a suturing catheter. The sheath includes proximal and distal ends, a window in a sidewall of the distal end and a balloon generally opposite the window. The suturing catheter includes proximal and distal ends, a suture carrier rotatably mounted to the distal end, and a capture device. During use, the sheath is advanced into a vessel and the balloon is expanded to stabilize the window against a vessel wall. The suturing catheter is advanced into the sheath, and a needle on the suture carrier is directly outwardly through the window along a curved path such that the needle passes through the wall and reenters the vessel. A suture on the needle is captured by the capture device and withdrawn from the vessel. A knot is advanced over the suture to secure the suture to the vessel wall.

### FIELD OF THE INVENTION

The present invention relates generally to apparatus and methods for delivering sutures, and, more particularly, to catheter devices for delivering sutures at remote locations within a patient's body, for example, during aneurysm repair using stent-grafts, heart valve repair or replacement, cardiac defect repair, ulcer repair, gastric volume reduction, and/or anti-reflux procedures performed on the gastro-esophageal junction.

### BACKGROUND

Since the advent of minimally invasive surgery, numerous devices have been suggested to replace the role that sutures and ligatures have traditionally played in open surgery. For example, various staples or clips have been suggested for general surgical applications, such as securing ends of a tubular graft within an aneurysm, e.g., within the abdominal aorta. As vascular surgery has transitioned to endovascular surgery and interventional radiology, such devices do not adequately replace sutures.

For example, when a tubular graft is implanted within an abdominal aneurysm during an open surgical procedure, ends of the graft may be secured to the ends of the retained vasculature using sutures. During endovascular repair of abdominal aneurysms, however, the ends of the graft are generally secured using a stent, e.g., an expandable tubular prosthesis, which may include external barbs to enhance fixation of the graft. Often, stents fail to adequately secure the ends of the graft, resulting in a phenomenon known as a “Type I endoleak,” i.e., when blood leaks around the stent-graft into the aneurysm cavity. This type of leak may lead to rupture of the aneurysm, re-operation, and/or increased risk of death of the patient.

### SUMMARY OF THE INVENTION

The present invention is directed to apparatus and methods for delivering sutures, and, more particularly, to catheter devices for delivering sutures at remote locations within a patient's body and to methods for using such devices. The apparatus and methods may be used to deliver sutures, for example, during aneurysm repair using stent-grafts, heart valve repair or replacement, gastric volume reduction, ulcer repair, anti-reflux procedures performed on the gastro-esophageal junction, to ligate a bleeding site during a colonoscopy, and/or other procedures.

In accordance with one embodiment, an apparatus is provided for delivering a suture within a patient's body that includes an elongate tubular member including a proximal end, and a distal end sized for introduction into a body lumen. A suture or needle carrier including a tip is rotatably mounted to the distal end. The suture carrier may be rotatable about an axis that is transverse, e.g., substantially perpendicular, to a longitudinal axis of the tubular member. In one embodiment, the suture carrier may be rotatable in a first direction such that the tip travels outwardly from a side of the distal end along a curved path until the tip reenters the distal end, and rotatable in a second opposite direction for retracting the tip back into the distal end along the curved path. A capture device may be positioned on the distal end for capturing a suture carried on the tip after the suture carrier is rotated in the first direction.

In an exemplary embodiment, a needle may be carried on the tip, and a suture may extend from the needle through the tubular member to the proximal end. The needle may include a sharpened point extending from the tip and/or a feature for releasably securing the needle to the tip of the suture carrier. In another embodiment, the tip of the suture carrier may be sharpened, and the suture carrier may include an element for releasably carrying a suture thereon.

In accordance with another embodiment, an apparatus is provided for delivering a suture within a patient's body that includes a delivery sheath and a suturing catheter. The delivery sheath may include a proximal end, a distal end sized for introduction into a body lumen, and a lumen extending between the proximal and distal ends. In addition, the delivery sheath may include a window in a sidewall of the distal end and an expandable member on the distal end generally opposite the window for directing the window against a wall of a body lumen within which the expandable member is expanded.

The suturing catheter may be an elongate tubular member including a proximal end, a distal end sized for introduction into the delivery sheath, and a suture carrier rotatably mounted to the distal end. The suturing catheter may be advanced into the delivery sheath until the suture carrier is disposed adjacent the window. In one embodiment, the suture carrier may be rotatable in a first direction such that the tip travels outwardly through the window along a curved path, and rotatable in a second opposite direction for retracting the tip back into the distal end along the curved path. A capture device may be positioned on one of the sheath and the suturing catheter for capturing a suture carried on the suture carrier after the suture carrier is rotated in the first direction.

In accordance with still another embodiment, a method is provided for delivering a suture at a remote location within a patient's body. Initially, a distal end of a tubular member may be advanced into a body lumen. A needle may be directly outwardly from the distal end along a curved path such that the needle penetrates a wall of the body lumen, passes through the wall, and reenters the body lumen, the needle carrying one end of a suture. The one end of the suture may be captured, and withdrawn from the body lumen, e.g., using a capture device. A knot may be advanced over the suture into the body lumen to secure the suture to the wall of the body lumen, and the suture may be severed to leave the knot in the body lumen.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a perspective view of an exemplary embodiment of an apparatus for delivering a suture into tissue that includes a delivery sheath, a suturing catheter, and a knot pusher.

FIG. 2 is a cross-sectional side view of a distal end of the delivery sheath of FIG. 1.

FIG. 2A is a cross-section of the distal end of the delivery sheath, taken along line 2A-2A in FIG. 2.

FIGS. 3A-3D are cross-sectional views of a distal end of the suturing catheter of FIG. 1, showing movement of a suture carrier thereon.

FIG. 4 is a perspective detail of the distal end of the suturing catheter in the position shown in FIG. 3A.

FIGS. 4A and 4B are cross-sections of the suturing catheter of FIGS. 3A and 4, taken along lines 4A-4A and 4B-4B, respectively.

FIG. 5 is a perspective view of an exemplary embodiment of a needle that may be carried by the suture carrier of FIGS. 3A-3D.

FIG. 6 is a perspective detail of a distal end of another embodiment of a delivery sheath.

FIGS. 7A-7C are side views of another embodiment of a suture carrier, including a pocket for releasably securing a suture to the suture carrier, that may be provided on a suturing catheter.

FIGS. 8A-8H are cross-sectional views of a patient's body, showing a method for delivering a suture into a wall of a body lumen.

FIG. 9A is a perspective view of a distal end of a loadable capture device, including a pair of opposing slots for supporting a noose.

FIG. 9B is a cross-section of the distal end of the capture device of FIG. 9, taken along line 9B-9B.

FIGS. 9C and 9D are cross-sectional views of the distal end of the capture device of FIG. 9B, taken along lines 9C-9C and 9D-9D, respectively, and showing a noose supported thereby.

FIG. 10 is a partial perspective and cross-sectional view of a distal end of a suturing catheter and a capture device that is insertable into the suturing catheter.

### DETAILED DESCRIPTION OF THE PREFERRED EMBODIMENTS

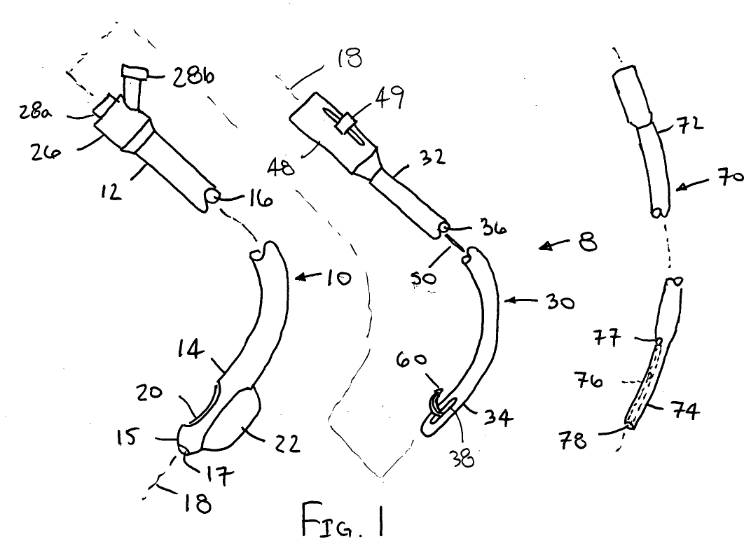

Turning to the drawings, FIGS. 1-4B show an exemplary embodiment of an apparatus 8 that includes a delivery sheath 10 and a suturing catheter 30 that may be introduced into the delivery sheath 10 for delivering a suture 50 into a tissue structure and/or a prosthesis at a remote location within a patient's body. For example, as explained further below, the apparatus 8 may be used to deliver a suture into a wall of a blood vessel or other body lumen (not shown), e.g., that may be secured using one or more knots (not shown) formed with the suture 50. In addition, the apparatus 8 may include one or more other components, e.g., a suture or other filament 50, a needle 60, a knot pusher 70, a cutter, one or more dilators, guidewires (all not shown), and the like, to provide a system or kit for delivering one or more sutures. Optionally, the system or kit may include other instruments for performing a procedure, e.g., a catheter or other device for delivering a stent-graft or other endoluminal prosthesis, a valve prosthesis, one or more additional suturing catheters, and the like (all not shown).

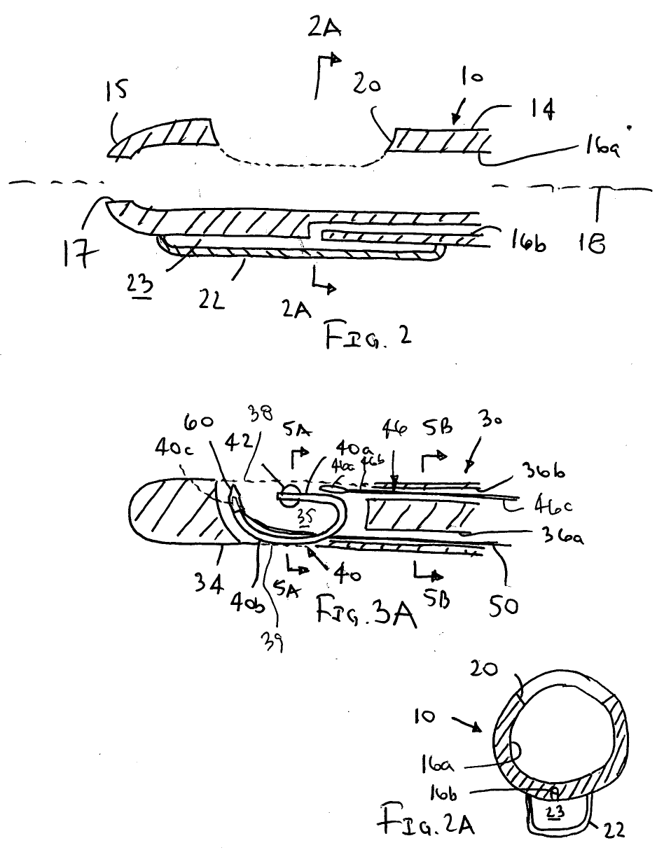

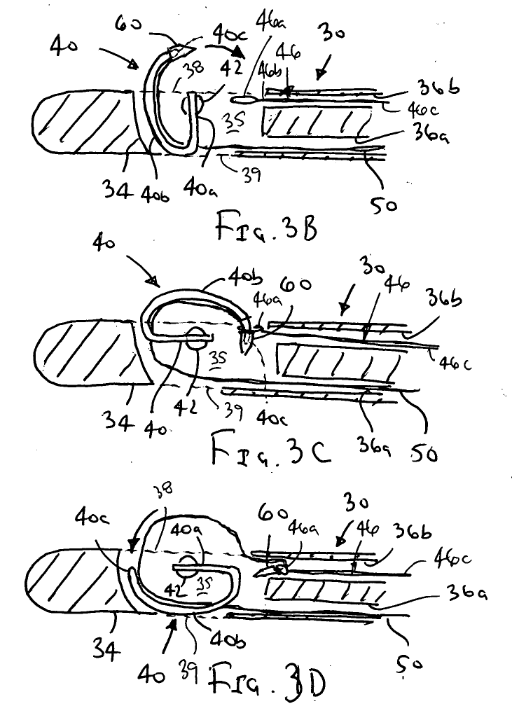

Generally, as shown in FIGS. 1 and 2, the sheath 10 is an elongate tubular member including a proximal end 12, and a distal end 14 sized and/or shaped for insertion into a body lumen of a patient (not shown), thereby defining a longitudinal axis 18 therebetween. The sheath may include one or more lumens 16 extending between the proximal and distal ends 12, 14. For example, as shown in FIGS. 2 and 2A, the sheath 10 may include an instrument lumen 16a and an inflation lumen 16b. The distal end 16 may terminate in a rounded, tapered, and/or other substantially atraumatic distal tip 15 including an opening 17 communicating with the instrument lumen 16a.

Alternatively, the sheath 10 may include a separate guidewire lumen (not shown) that may extend from the proximal end 12 to the opening 17, and the instrument lumen 16b may terminate within the distal end 14. In yet another alternative, the instrument lumen 16b or a separate guidewire lumen may terminate proximal to the distal end 14, e.g., at a side wall opening (not shown) located a predetermined distance from the distal tip 15, to provide a “rapid-exchange” lumen.

The sheath 10 may be formed from plastic, metal, or composite materials, e.g., a plastic material having a braided, coiled, or other wire core, which may preventing kinking or buckling of the catheter 12 during advancement. Optionally, the sheath 10 may have variable flexibility along its length. For example, the distal end 14 may be substantially flexible to facilitate insertion through tortuous anatomy. The distal end 14 may be sized and/or shaped for introduction into a body lumen, e.g., having a diameter between about two and nine millimeters (2-9 mm). The proximal end 12 may be substantially flexible or semi-rigid, e.g., having sufficient column strength to facilitate advancing the distal end 14 through a patient's vasculature by pushing on the proximal end 12.

As shown in FIG. 1, the sheath 10 may include a handle 26 on the proximal end 12, e.g., to facilitate manipulating the apparatus 10. The handle 26 may include one or more ports 28 communicating with respective lumens 16 within the sheath 10. For example, port 28a may communicate with instrument lumen 16a, and side port 28b may communicate with inflation lumen 16b. The port(s) 28 may include one or more seals and/or connectors. For example, port 28a may include a hemostatic seal, allowing one or more instruments, such as the suturing catheter 30, to be introduced into the instrument lumen 16a while preventing blood or fluid from escaping proximally from the port 28a. The side port 28b may include a luer lock connector and the like (not shown), e.g., to allow a syringe or other source of inflation media and/or vacuum (also not shown) to be coupled to the inflation lumen 16b, e.g., via tubing and the like (also not shown).

The handle 26 may be formed from plastic, metal, or composite material, e.g., providing an outer casing, which may be contoured or otherwise shaped to ease manipulation. The proximal end 12 of the sheath 10 may be attached to the handle 26, e.g., by bonding, cooperating connectors, interference fit, and the like. Optionally, if the sheath 10 includes any actuatable components (such as a mechanically expandable member, not shown) on the distal end 14, the handle 26 may include one or more actuators, such as one or more slides, dials, buttons, and the like (not shown), for activating, deactivating, and/or otherwise manipulating the component(s) on the distal end 16 from the proximal end 14.

With continued reference to FIGS. 2 and 2A, the distal end 14 may include a window 20 in a sidewall of the sheath 10 that communicates with the instrument lumen 16a. In addition, the distal end 14 may also include a balloon 22 or other expandable member, e.g., on the sidewall generally opposite to the window 20. An interior 23 of the balloon 22 may communicate with the inflation lumen 16b such that the balloon 22 may be expanded from a contracted condition (such as that shown in FIG. 2) to an enlarged condition (such as that shown in FIGS. 1 and 2A) when inflation media is delivered into the interior 23 via the inflation lumen 16b, as explained further below.

As best seen in FIG. 2A, the window 20 may extend partially around a circumference or other periphery of the sheath 10, e.g., not more than about two hundred twenty degrees (220°) or less than about one hundred eighty degrees (180°) around the circumference. The window 20 may have a predetermined length, e.g., between about two and ten millimeters (2-10 mm), to provide a working window for accessing an adjacent tissue structure, e.g., a wall of a blood vessel or other body lumen (not shown), using an instrument, such as the suturing catheter 30. Alternatively, a plurality of windows (not shown) may be disposed adjacent one another, e.g., spaced apart from one another axially along the distal end 14.

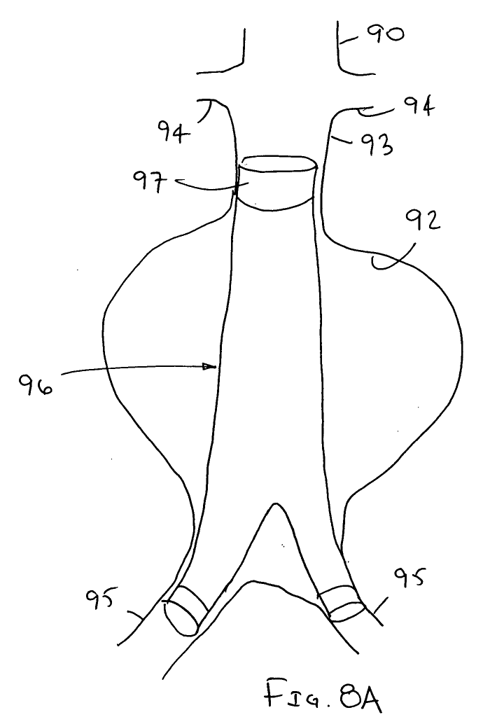

In yet another alternative, shown in FIG. 6, a delivery sheath 10′ may be provided, otherwise similar to other embodiments described herein, that includes an open distal end 14′ defining a window 20.′ For example, the distal end 14′ may be formed from a tube that has a portion of the sidewall removed to define the window 20′ and provide a substantially atraumatic distal tip 15.′ A balloon 22′ may be provided on the distal end 14′ generally opposite the window 20′ that may be expanded from a contracted condition (shown solid) to an enlarged condition (shown in phantom). The window 20′ may communicate with an instrument lumen 16a′ extending to a proximal end (not shown) of the sheath 10.′

Returning to FIGS. 2 and 2A, the balloon 22 may be a flexible membrane that is attached or otherwise secured to the distal end 14 of the sheath 10. For example, outer edges of the membrane may be bonded to the outer surface of the sheath 10, e.g., using an adhesive, sonic welding, fusing, and the like. Alternatively, the outer edges of the membrane may be at least partially embedded in the wall of the sheath 10 and/or one or more outer edges, e.g., on proximal and/or distal ends of the balloon 22, may be secured to the distal end 14 using a band (not shown) extending around the periphery of the sheath 10. Thus, the balloon 22 may have a profile in the contracted condition that conforms substantially to an outer surface of the distal end 14, thereby minimizing a profile of the distal end 14 to facilitate delivery through a patient's vasculature or other body lumen. In another alternative, the balloon 22 may be a substantially enclosed body (not shown) secured to the distal end 14 of the sheath 10, e.g., including an opening communicating with the inflation lumen 16b.

The balloon 22 may be formed from substantially elastic material such that the size of the balloon 22 in the enlarged condition is proportional to a volume and/or pressure of the inflation media delivered into the interior 23. In addition, the balloon 22 may be substantially noncompliant. Thus, a single balloon 22 may be expanded to a range of sizes, yet have sufficient integrity to substantially anchor the distal end 14 of the sheath 10 within a body lumen, e.g., to stabilize the window 20 against a wall of the body lumen, as explained further below. Alternatively, the balloon 22 may be formed from substantially inelastic material such that the balloon 22 may be collapsed against the distal end 14 in the contracted condition. When inflation media is delivered into the interior 23, the balloon 22 may expand to a predetermined shape and/or size, e.g., extending a predetermined distance from the outer surface of the sheath 10. In this alternative, the balloon 22 may be used to anchor the sheath 10 within body lumens having a range of sizes corresponding to the size of the balloon 22 in the enlarged condition. Exemplary materials for the balloon 22 may include latex, silicone, polyurethane, polyester, PET, nylon, and the like.

In another alternative, a mechanically expandable member may be provided on the distal end 14. For example, a frame may be provided within or otherwise carrying a membrane (not shown), e.g., provided from metal, such as stainless steel or Nitinol, plastic, such as a thermoplastic, or composite material, that may be expanded from a contracted condition for delivery to an enlarged condition. The frame may be actuated to expand away from the distal end to the enlarged condition to substantially anchor or otherwise stabilize the distal end 14 at a target location, as explained further below. Instead of an inflation lumen, the sheath may include one or more actuator lumens carrying one or more cables or other members that may be controlled from the handle 26 for expanding and/or collapsing the frame. Optionally, the expandable member (whether inflatable or mechanically expandable) may include a passage extending axially therethrough (not shown) or may be otherwise shaped to allow blood or other fluid flow through or around the expandable member even in the enlarged condition.

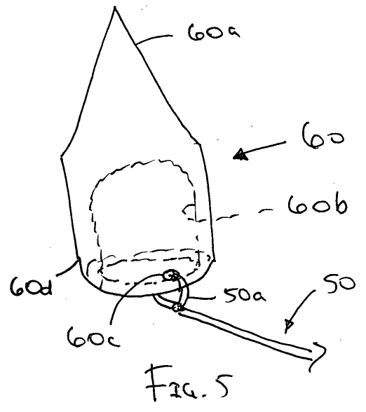

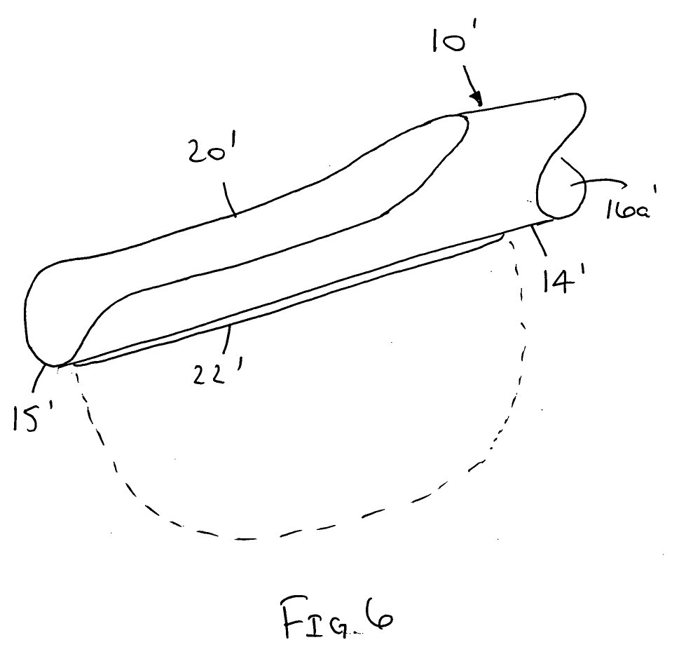

Returning to FIG. 1, the suturing catheter 30 is an elongate tubular member including a proximal end 32, a distal end 34, and a plurality of lumens 36 extending therebetween, e.g., generally parallel to the longitudinal axis 18. In the embodiment shown in FIG. 4B, the suturing catheter 30 may include a suture lumen 36a, a capture device lumen 36b, and one or more actuator lumens 36c. The suturing catheter 30 may be constructed similar to the sheath 10, e.g., including similar or different materials and/or methods along its length. In addition, the suturing catheter 30 may include a handle 48 on the proximal end 32, which may include one or more actuators (an exemplary actuator 49 being shown) for activating components on the distal end 34 of the suturing catheter 30, also similar to the sheath 10.

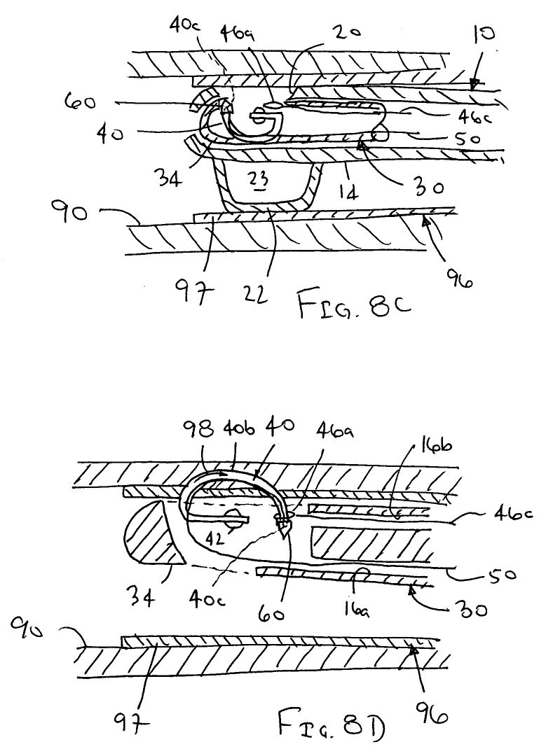

The distal end 34 of the suturing catheter 30 may be sized and/or shaped to be received in the instrument lumen 16a of the sheath 10 such that the distal end 34 may be advanced into the distal end 14 of the sheath 10, e.g., adjacent the window 20, as shown in FIGS. 8A-8H and explained further below. Optionally, the suturing catheter 30 and/or sheath 10 may include one or more stops or other features for limiting distal advancement of the suturing catheter 30 into the sheath 10. For example, as shown in FIG. 2, the distal end 14 of the sheath 10 may taper inwardly at a location distal to the window 20, thereby preventing the distal end 34 of the suturing catheter 30 from being advanced beyond the window 20.

Alternatively, one or more tabs or other features (not shown) may extend inwardly from the distal end 14 into the instrument lumen 16a against which the distal end 34 of the suturing catheter 30 may abut to prevent additional distal movement. In yet another alternative, one or more tabs or other features (also not shown) may extend radially outwardly from the distal end 34 of the suturing catheter 30, e.g., that may be slidably received in a track, pocket, and the like (also not shown) in the sheath 10 for limiting distal movement.

In addition, if desired, the distal end 34 of the suturing catheter 30 and/or the instrument lumen 16a may be “keyed” such that the distal end 34 of the suturing catheter 30 is received within the distal end 14 of the sheath 10 in a predetermined angular orientation about the longitudinal axis 18. For example, the distal end 34 of the suturing catheter 30 and the instrument lumen 16a may have complementary cross-sections, e.g., noncircular or other asymmetrical cross-sections, such that the distal end 34 of the suturing catheter 30 is aligned with the window 20 in a desired manner, as explained further below. In addition or alternatively, the instrument lumen 16a may include one or more tracks, ramps, or guides (not shown) that orient the distal end 34 of the suturing catheter 30 as it is advanced into the distal end 14 of the sheath 10 and/or limit distal movement.

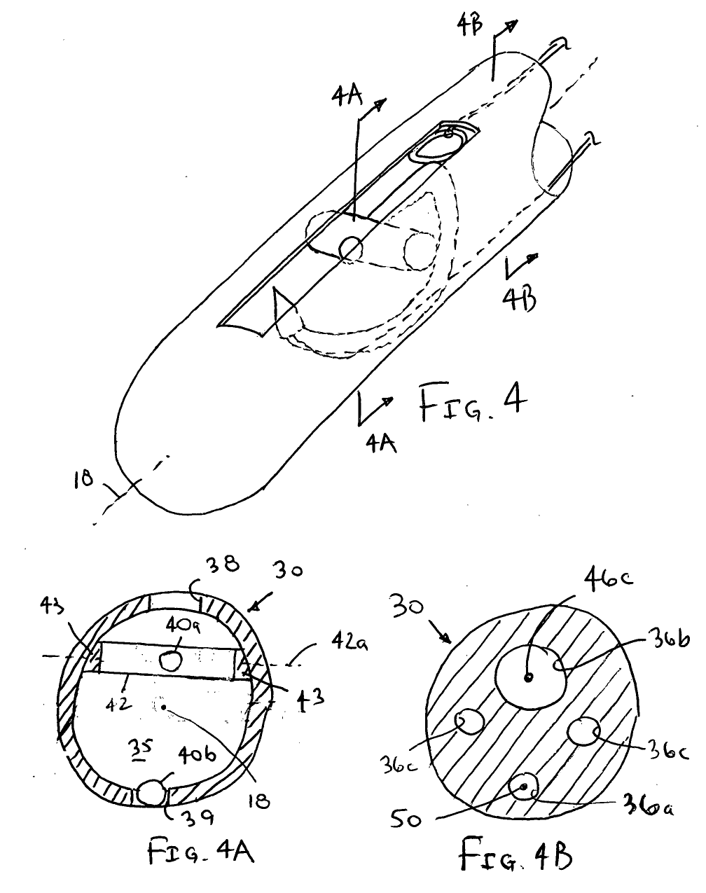

With additional reference to FIGS. 3A-4B, the distal end 34 of the suturing catheter 30 may include a suture carrier 40 rotatably mounted to the distal end 34. As shown in FIGS. 3A and 4, the distal end 34 may include a slot 38 located adjacent the suture carrier 40. The suture carrier 40 may be disposed within an interior region 35 of the distal end 34, and the slot 38 may extend longitudinally along the distal end 34 overlying the suture carrier 40 and/or the interior region 35. Optionally, the wall of the distal end 34 may be at least partially removed opposite the slot 38, e.g., to provide a recess or opening 39 to accommodate the suture carrier 40. For example, the opening 39 may allow an overall diameter or size of the distal end 34 to be reduced for a given size suture carrier 40, i.e., by removing all or a portion of the wall at the opening 39, thereby allowing the suture carrier 40 to nest into the wall of the distal end 34.

Generally, the suture carrier 40 includes a base 40a coupled to an axle 42, and a curved support 40b extending from the base 40a, the curved support 40b terminating in a tip 40c. For example, the suture carrier 40 may be a substantially rigid wire, rod, or tube, e.g., having an outer diameter or other cross-section between about 0.25-0.75 mm, bent or otherwise formed into the shape shown. The curved support 40b may define an arc, e.g., a portion of a circle or ellipse, for example, an arc defining between about 120-220 degrees of a circumference of a circle. In addition, the base 40a may fix the curved support 40b at a predetermined radius or other distance from an axis of rotation 42a defined by the axle 42, e.g., between about one to eight millimeters (1-8 mm). As best seen in FIG. 4A, the axle 42 may be offset transversely from the longitudinal axis 18, e.g., towards the slot 38, by between about one to five millimeters (1-5 mm). For example, the axle 42 may be mounted within about 0.5-1.5 mm of the side wall adjacent the slot 38, e.g., to dispose the tip 40c of the suture carrier 40 (or a needle 60 carried thereon), immediately adjacent or within the slot 38. Thus, when the suture carrier 40 is rotated, a curved path may be defined having a predetermined radius about the axis 42a, at least a portion of the curved path extending transversely beyond a sidewall of the distal end 34.

As shown in FIGS. 3A and 4, the suture carrier 40 may be disposed initially within the distal end 34 adjacent the slot 38 with the base 40a extending substantially parallel to the longitudinal axis 18 and the tip 40c extending transversely immediately adjacent or within the slot 38. For example, in the initial position, the tip 40c may define an angle between about seventy and one hundred ten degrees (70-110°), relative to the longitudinal axis 18. In one embodiment, the tip 40c may be oriented substantially perpendicularly (e.g., at an angle of about eighty degrees (80°) or about ninety degrees (90°)). This configuration may facilitate penetrating or otherwise driving the tip 40c (or a needle 60 carried thereon) into tissue or other adjacent structures. In addition or alternatively, this configuration may maximize the “bite” or depth of penetration of the suture carrier 40 into tissue or other adjacent structures. For example, with the axle 42 disposed immediately adjacent the sidewall, the radius of the suture carrier 40 may approach the diameter of the suturing catheter 30. Thus, the distance that the tip 40c of the suture carrier 40 may extend transversely from the distal end 34 may be maximized for a given diameter of the suturing catheter 30.

Optionally, to dispose the tip 40c immediately adjacent or within the slot 38, the tip 40cmay include a substantially straight section (not shown),e.g., having a length between about one and three millimeters (1-3 mm), that extends from the curved support 40b. This delivery configuration may facilitate advancing the suturing catheter 30 into the sheath 10 and/or into a body lumen (not shown), without exposing the suture carrier 40, a suture 50, and/or needle 60 carried on the tip 40c. As shown in FIGS. 3B and 3C, the suture carrier 40 may be rotatable about the axis 42a in a first direction such that the tip 40c travels outwardly from a side of the distal end 34, e.g., through the slot 38, and along the curved path (see FIG. 3B). When the suture carrier 40 is completely rotated in the first direction, the tip 40c may reenter the distal end 34 (see FIG. 3C), e.g., through the slot 38.

Complete rotation in the first direction may involve the suture carrier 40 rotating less than three hundred sixty degrees (360°) or not more than about two hundred seventy degrees (270°), e.g., between about 150-270 degrees. The suture carrier 40 may also be rotatable about the axis 42a in a second direction, e.g., opposite the first direction, for retracting the tip 40c back into the distal end 34 along the curved path, as shown in FIG. 3D. Alternatively, the distal end 34 may include multiple openings (not shown) instead of the slot 38, e.g., one opening at the location where the suture carrier 40 exits the distal end 34 and another opening where the tip 40c reenters the distal end 34.

The suture carrier 40 may be formed from stainless steel or other substantially rigid material, providing sufficient column and/or other structural strength to allow the tip 40c and curved support 40b to be advanced through tissue along the curved path, as explained further below. The base 40a may be mounted to the axle 42, which, in turn, may be mounted within the distal end 34, yet allowing the suture carrier 40 to rotate about the axis 42a.

In one embodiment, shown in FIG. 4A, the axle 42 may be rotationally mounted to one or more hubs or other supports 43 extending inwardly from a side wall of the suturing catheter 30, and the base 40a may be fixed to the axle 42. For example, the axle 42 may be a tubular segment that slidably receives the hubs 43 therein. Alternatively, the axle 42 may be a solid or hollow rod, and the hubs 43 may be annular ridges or circular recesses that rotatably receive ends of the axle 42 therein.

The base 40a may be secured to the axle 42, e.g., force fit or otherwise inserted through an opening or pocket in the axle 42. In addition or alternatively, the base 40a may be fixed to the axle 42 using an adhesive, sonic welding, and the like. As shown in FIGS. 3A-3D, the suture carrier 40 may be oriented such that the tip 40c exits the distal end 34 at a location distal to a location where the tip 40c reenters the distal end 34 (i.e., such that the first direction is clockwise). Alternatively, if desired, the suture carrier 40 may be mounted such that the tip 40cexits the distal end 34 at a location proximal to a location where the tip 40c reenters the distal end 34 (i.e., such that the first direction is counterclockwise, not shown).

With additional reference to FIG. 1, the suture carrier 40 may be actuated from the proximal end 32 of the suturing catheter 30, e.g., using one or more actuators on the handle 48, to cause the suture carrier to rotate in the first and/or second directions. For example, a suture, wire, cable, or other advancement filament (not shown) may be wound around a portion of the axle 42. The advancement filament may extend into a lumen, such as actuator lumen 36c shown in FIG. 4B, through the proximal end 32 and into the handle 48. A first actuator on the handle 48 (such as slide 49 shown in FIG. 1) may be coupled to the advancement filament such that, when the first actuator is manipulated (e.g., by pulling the slide 49 proximally), the advancement filament is pulled proximally, thereby causing the axle 42 and suture carrier 40 to rotate in the first direction as the advancement filament at least partially unwinds from the axle 42.

In one embodiment, this action may cause a retraction filament (also not shown) to wrap at least partially around another portion of the axle 42. For example, the advancement and retraction filaments may be wound around opposite ends of the axle 42, e.g., on either side of the base 40a of the suture carrier 40. The retraction filament may also extend through a lumen, e.g., another actuator lumen 36c, through the proximal end 32 and into the handle 48 where the retraction filament may be coupled to a second actuator. When the second actuator is manipulated, the retraction filament may be pulled proximally, thereby causing the axle 42 and suture carrier 40 to rotate in the second direction as the retraction filament at least partially unwinds from the axle 42. Optionally, a single actuator may be provided that is coupled to the advancement and retraction filaments for selectively rotating the suture carrier 40 in either the first or the second direction. For example, the advancement filament may have sufficient column strength to be pushed distally for rotating the axle 42 in the second direction.

Alternatively, a gear mechanism (not shown) may be provided that is coupled to axle 42 and/or to an actuator on the handle 48. For example, a cable that rotates about the longitudinal axis may be coupled to the axle 42 by one or more gears. When the cable is rotated in a first direction around the longitudinal axis 18, e.g., using an actuator on the handle 48, the axle 42 may be rotated in the first direction. When the cable is rotated in a second opposite direction around the longitudinal axis 18, the axle 42 may be rotated in the second direction. It will be appreciated that other mechanisms may be provided for rotating the axle 42 in response to an actuator on the handle 48.

In addition, with particular reference to FIGS. 3A-4, the suturing catheter 30 may also include a capture or retention device 46 for capturing a suture 50 carried on the tip 40c of the suture carrier 40, e.g., after the suture carrier 40 is completely rotated in the first direction. In the embodiment shown, the capture device 46 includes a noose 46a on one end 46b of a filament 46cthat extends proximally from the distal end 34. The filament 46c may extend through the capture device lumen 36b, as shown in FIG. 4B, into the handle 48 and/or out of the suturing catheter 30.

The noose 46a may be suspended or otherwise supported within the distal end 34 of the suturing catheter 30 adjacent to the slot 38, e.g., using one or more support structures (not shown) within the suturing catheter 30. The noose 46a may have a sufficiently large diameter or other configuration to allow the tip 40c of the suture carrier 40 and/or needle 60 to pass freely through the noose 46a when the tip 40c enters the slot 38, e.g., as shown in FIG. 3C.

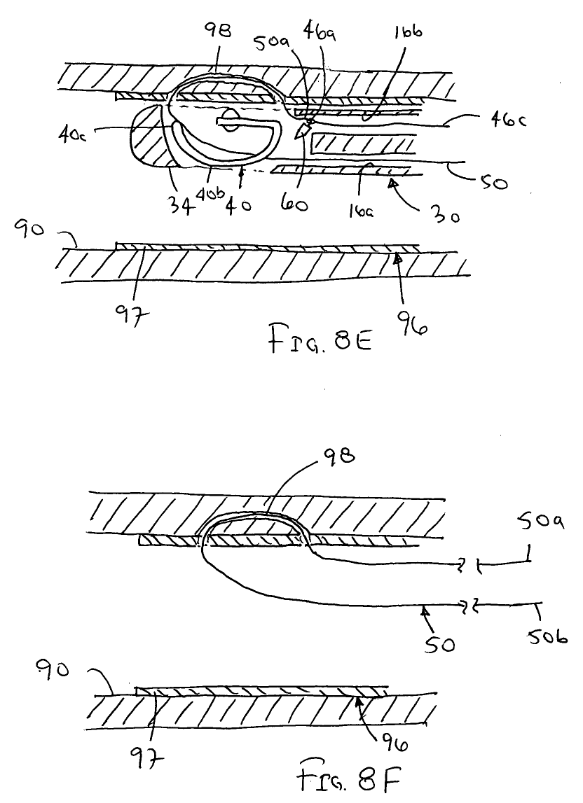

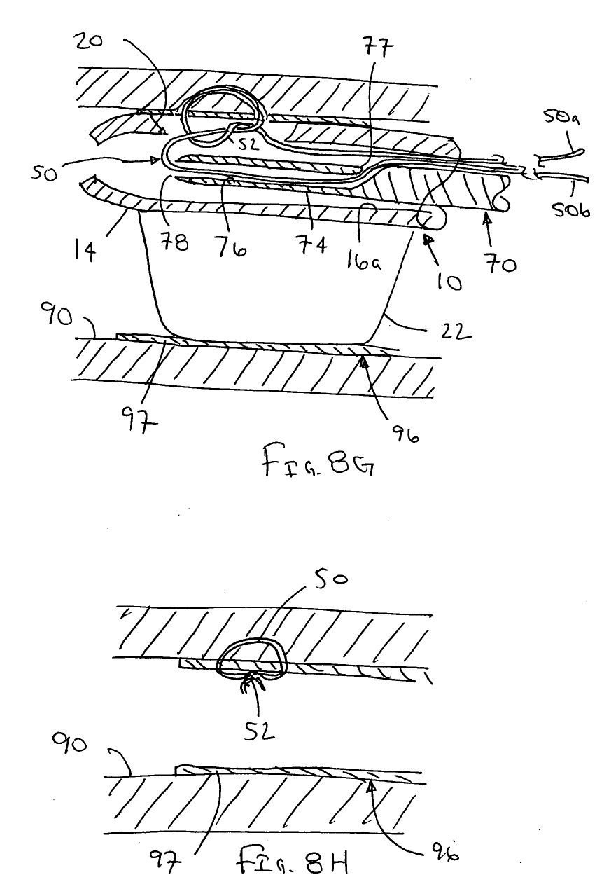

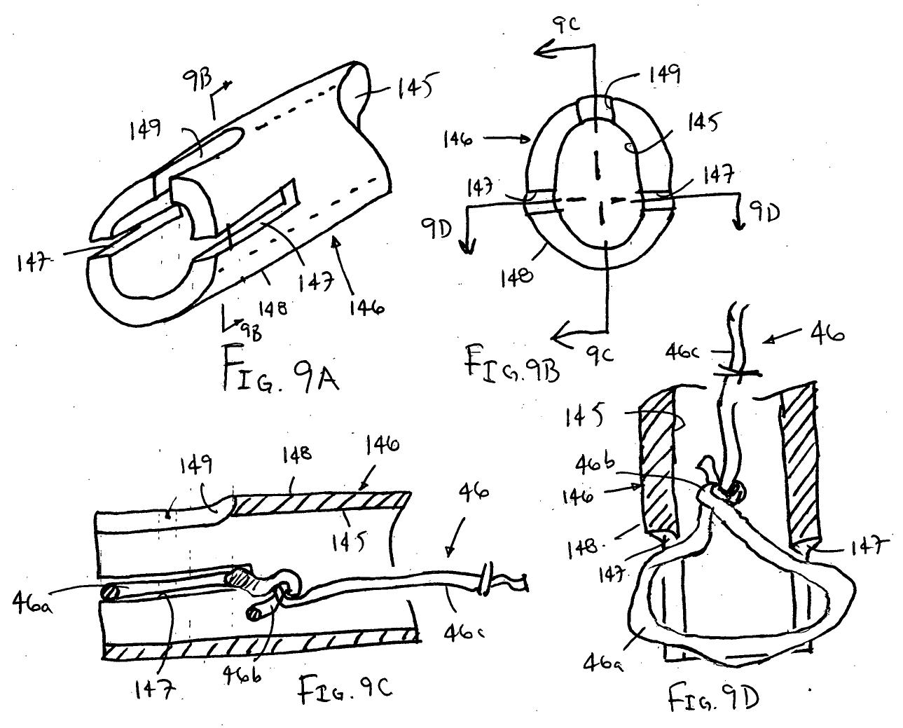

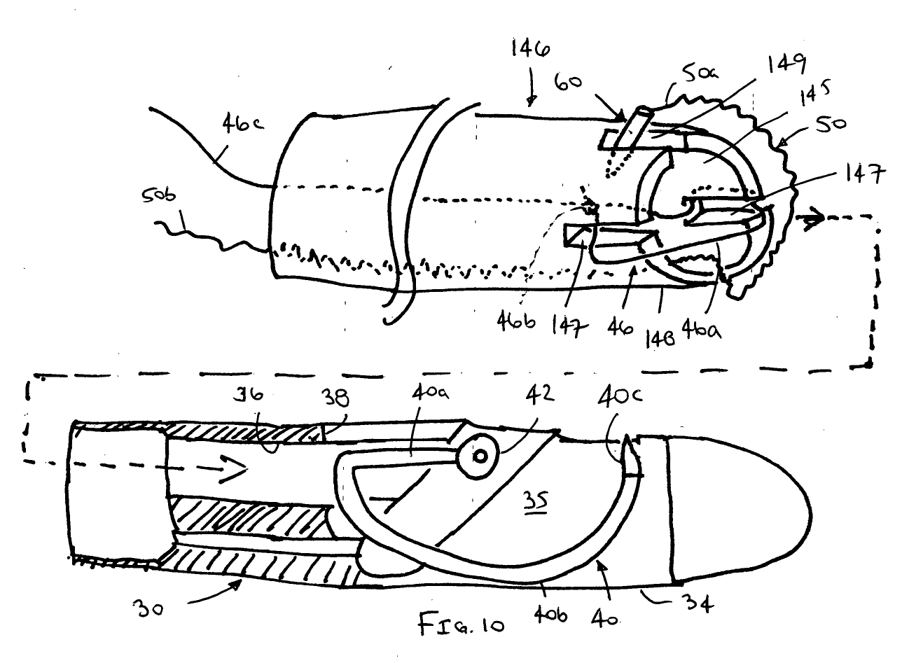

Alternatively, as shown in FIGS. 9A-9D and 10, a loadable capture device 146 may be provided within a suturing catheter 30, such as those described elsewhere herein. The capture device 146 may include a proximal end (not shown), a distal end 148, and one or more lumens 145 extending therebetween. As shown in FIG. 10, the distal end 148 of the capture device 146 may be sized and/or shaped to be introduced into a lumen 36 of the suturing catheter 30. The capture device 146 may include a pair of opposing slots or recesses 147, e.g., formed in a wall of the distal end 148. As shown in FIGS. 9C and 9D, the slots 47 may receive a portion of a noose 46a therein, e.g., to substantially support the noose 46a fully open adjacent the slot 38.

The capture device 146 may be provided within the suturing catheter 30 such that the noose 46a is disposed within the distal end 34 adjacent the slot 38, e.g., when the apparatus 8 is assembled during manufacturing. Alternatively, one or more capture devices 146 may provided separately from the suturing catheter 30 that may be loaded into the suturing catheter 30 before or during a procedure. For example, immediately before a procedure, a first capture device 146 may be loaded into or otherwise provided within the suturing catheter 30 and used to deliver a first suture 50, as described further elsewhere herein.

Optionally, the first capture device 146 may be used to load a first needle and/or suture into a suturing catheter 30 if the suturing device 30 does not initially have a needle 60 and/or suture 50 preloaded therein. For example, as shown in FIGS. 9A-9D, the capture device 146 may include a slot or pocket 149 for receiving a needle 60. As shown in FIG. 10, a needle 60 carrying a suture 50 may be loaded through the capture device 146 such that the suture 50 extends to the proximal end (not shown) of the capture device 146, and the needle 60 is removably but securely received in the pocket 149. The capture device 146 may then be advanced into the suturing catheter 30 to advance the needle 60 and/or suture 50 through the suturing catheter 30 to the distal end 34. The needle 60 may then be placed over or otherwise onto the tip 40c of the suture carrier 40, as described elsewhere herein. Alternatively, a suture 50 may be loaded directly onto the suture carrier 140, also as described elsewhere herein.

In addition, the capture device 146 may allow multiple sutures to be delivered using a single suturing catheter 30. For example, after delivering a first suture 50, the capture device 146 may be removed from the suturing catheter 30, and another capture device 146 may be inserted into the suturing catheter 30. The capture device 146 may carry another needle 60 and/or suture 50, which may be loaded onto the suture carrier 40, as described above.

Alternatively, a single capture device 146 may be used to deliver multiple sutures. For example, after delivering a first suture, the capture device 146 may be removed from the suturing catheter 30. A replacement noose 46a and/or filament 46c may be directed through the lumen 145 of the capture device 146, e.g., such that the noose 46a is supported by the slots 147 and the filament 46c extends to the proximal end (not shown) of the capture device 146. An additional needle 60 and/or suture 50 may be loaded into the capture device 146, e.g., such that the needle 60 is received in the pocket 149 and the suture 50 extends through the lumen 145, as described above. The capture device 146 may then be directed back into the suturing catheter 30 until the noose 46a, and needle 60 and/or suture 50 are disposed within the distal end 34 of the suturing catheter 30. The needle 60 and/or suture 50 may be loaded onto the needle carrier 40, 140, as described above, and used to deliver the additional suture 50.

The noose 46a may be formed from a loop, e.g., using a slipknot tied around the end 46bof the filament 46c, such that the noose 46a may be constricted or otherwise tightened around the suture carrier 40, e.g., to capture a suture 50 carried thereby, as explained further below. For example, as shown in FIG. 3C, with the tip 40c disposed through the noose 46a, the filament 46c may be pulled proximally, thereby tightening the noose 46a around the suture carrier 40b. When the filament 46c is pulled further, the suture 50 and/or needle 60 may be pulled off of the tip 40c and into the capture device lumen 36b, as shown in FIG. 3D and described further below. In addition, if the noose 46a is suspended within a support structure, such as the slots 147 shown in FIGS. 9A-9D, the noose 46a may be pulled inwardly out of the slots 147 when the noose 46a is tightened or the filament 46c is pulled proximally. Alternatively, the suture 50 and/or needle 60 may be pulled off of the tip 40c when the suture carrier 40 is retracted back along the curved path.

The noose 46a and/or filament 46c may be a single segment of monofilament or multi-filament, e.g., made from nylon, silk, polyester, stainless steel, Nitinol, or other material, that extends to the proximal end 32 of the suturing catheter and/or into or through the handle 48. The filament 46c may have sufficient tensile strength to be pulled without substantial risk of breaking. Alternatively, the filament 46c may extend into an intermediate portion of the suturing catheter 30, where the filament 46c may be coupled to another cable or element (not shown) that extends to the proximal end 32. In one embodiment, the handle 48 may include an actuator, e.g., similar to slide 49 shown in FIG. 1, that may be coupled to the filament 46c. When the actuator is activated (e.g., by sliding the slide 49 proximally), the filament 46c may be pulled proximally to tighten the noose 46a and/or draw the noose 46a proximally into the capture device lumen 36b.

In one embodiment, the filament 46c may be pulled a predetermined distance into the capture device lumen 36b to secure one end of the suture 50 to the suturing catheter 30 proximal and/or adjacent to the distal end 34. Alternatively, the filament 46c may be withdrawn proximally completely through the suturing catheter 30 until the end of the suture 50 is pulled completely out of the patient's body, as explained further below.

In an alternative embodiment, other snares or other capture devices may be provided, such as the snares disclosed at http://www.bostonscientific.com/med specialty/deviceCategoryList.jsp?task=tskCategoryList.jsp&sectionId=4&relId=7,334,2430, the entire disclosure of which is expressly incorporated by reference herein. In a further alternative, a micro-grasper or other tool (not shown) may be provided in the distal end 34 for grabbing or otherwise securing the needle 60 and/or suture 50.

Returning to FIG. 4, in an exemplary embodiment, the distal end 34 of the suturing catheter 30 may be formed or otherwise made as semi-cylindrical portions (not shown) that may be attached together, e.g., along a seam that intersects the slot 38. For example, each portion may include a hub 43 and part, e.g., half, of the slot 38. Optionally, the semi-cylindrical portions may include mating connectors, e.g., corresponding tabs and pockets, grooves, and the like (not shown) for facilitating assembly. With the semi-cylindrical portions separated, the axle 42 carrying the suture carrier 40 may be aligned with the hubs 43, and then the semi-cylindrical portions may be attached together, capturing the axle 42 therein. Optionally, the semi-cylindrical portions may be fixed together, e.g., using one or more of an adhesive, sonic welding, interference fit, and/or mating connectors at one or more locations along the seam.

The assembled distal end 34 may then be connected to a remainder of the suturing catheter 30, e.g., an intermediate portion extending to the proximal end 32, which may include the lumens 36 shown in FIG. 4B. The distal end 34 may be connected to the intermediate portion, for example, using mating connectors, an interference fit, a shrink tube or other surrounding band, adhesives, melting or otherwise fusing the ends, and the like. The filaments or other actuating components may be disposed within the distal end 34 and directed through the lumens 36 to the proximal end 32 of the suturing catheter 30, where the filaments may be. coupled to one or more actuators on the handle 48, e.g., using materials and methods known to those skilled in the art.

In the embodiment shown in FIG. 4, a needle 60 may be provided on the tip 40c of the suture carrier 40, e.g., during assembly of the suturing catheter 30. A suture 50 may be coupled or otherwise extend from the needle 60, e.g., into suture lumen 36a to the proximal end 32 of the suturing catheter 30. The needle 60 may be machined, laser cut, etched, or otherwise formed from substantially rigid material, such as surgical grade stainless steel. The suture 50 may be formed any conventional suture materials, e.g., nonresorbable materials, such as silk, polypropylene, polyester, nylon, steel wire, PTFE, FEP, and the like, or resorbable materials, such as poly-lactic acid (“PLA”), poly-glycolic acid (“PGA”), gut, and the like.

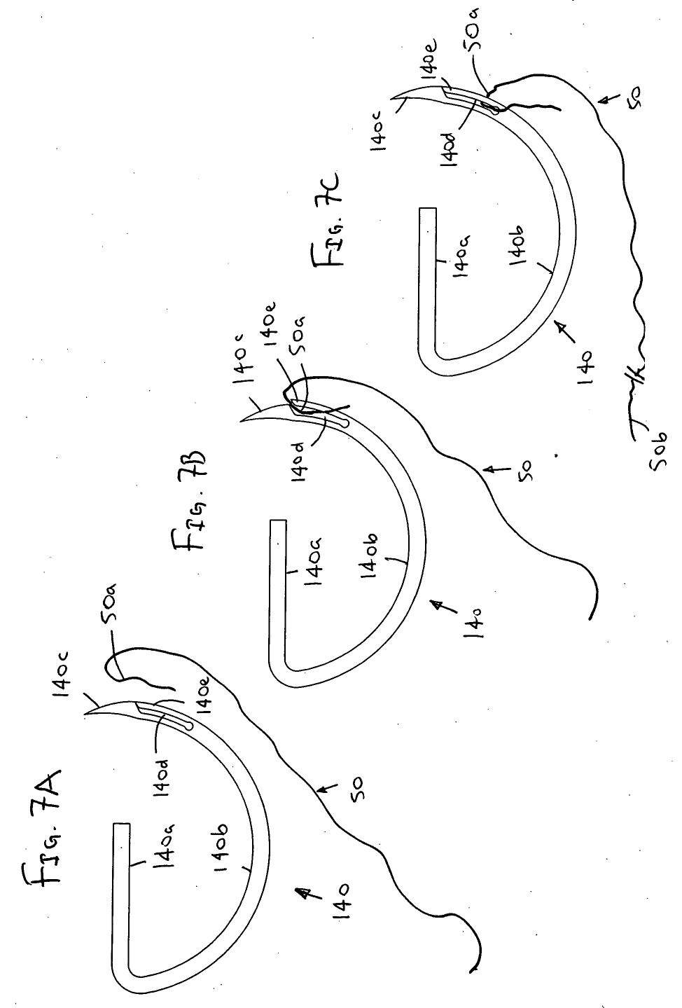

Turning to FIG. 5, in an exemplary embodiment, the needle 60 may include a sharpened point 60a on one end and a recess 60b in the other end 60d. The recess 60b may be defined by a cylindrical wall or by a plurality of fingers spaced apart around the circumference of the end 60dof the needle 60. The recess 60b may have a diameter and/or depth corresponding to the tip 40cof the suture carrier 40 such that the needle 60 may be placed stably on the tip 40c, yet be removed using a capture device, as described elsewhere herein. In an exemplary embodiment, the needle 60 may have an overall length of not more than about 0.250 inch (6 mm) and a diameter at the end 60d between about two and twelve millimeters (2-12 mm).

A first end 50a of the suture 50 may be secured to the needle 60, e.g., adjacent the recess 60b. For example, the needle 60 may include an eyelet 60c through which the first end 50a of the suture 50 may be directed and tied. Alternatively, the first end 50a of the suture 50 may be wrapped around a circumference of the needle 60 and tied (not shown). In another alternative, the first end 50a of the suture 50 may be attached using an adhesive, fusing, sonic welding, and the like, e.g., to attach the first end 50a to the needle 60 and/or to the suture 50 itself. A second end (not shown) of the suture 50 may be loose, e.g., extending through the suture lumen 36a of the suturing catheter 30, as shown in FIG. 4B, through the proximal end 30, and into or through the handle 48.

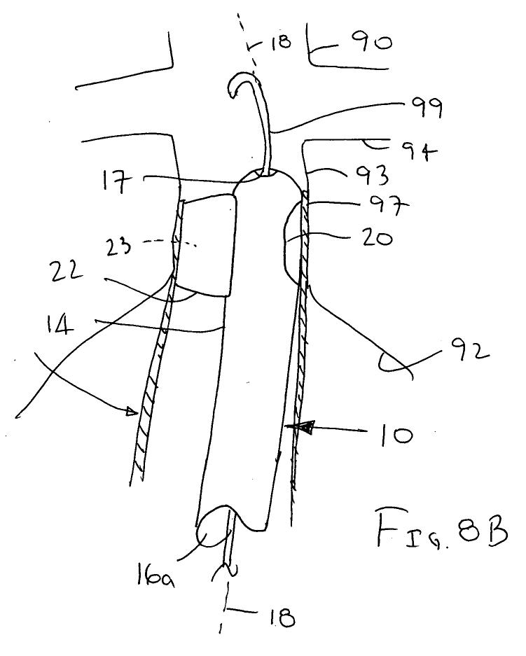

Turning to FIGS. 7A-7C, another embodiment of a suture carrier 140 is shown. Similar to the previous embodiment, the suture carrier 140 includes a base 140a, and a curved support 140b extending from the base 40a, the curved support 140b terminating in a tip 140c. Unlike the previous embodiment, the tip 140c is pointed or otherwise sharpened such that the tip 140c may be forced or otherwise advanced through tissue. In addition, the suture carrier 140 includes a pocket 140d adjacent the tip 140c for receiving a first end 50a of a suture 50 therein. Optionally, the suture carrier 140 may include a tongue 140e or other cover that at least partially covered the pocket 140d, e.g., to secure the first end 50a of the suture in the pocket 140d.

For example, the tongue 140e may be directed away from the pocket 140d, as shown in FIG. 7B, to allow the first end 50a of the suture 50 to be inserted into the pocket 140d. The tongue 140e may resiliently return towards the pockets 140d, as shown in FIG. 7C, thereby frictionally engaging the first end 50a of the suture 50 to the suture carrier 140. When the suture carrier 140 is rotated in the first direction (similar to that shown in FIGS. 3A and 3B), the first end 50a of the suture 50 may be maintained in a bottom of the pocket 140d, e.g., due to the tension experienced by the suture 50.

Similar to FIGS. 3C and 3D, the noose 46a may be pulled around the suture carrier 140 to secure the first end 50a of the suture 50 within the noose 46a. When the suture carrier 140 is retracted in the second direction, the noose 46a may sufficiently frictionally engage the first end 50a of the suture 50 to pull the suture 50 out of the pocket 140d, allowing the suture 50 to be pulled proximally out of the distal end of the suturing catheter, similar to FIG. 3D.

Returning to FIG. 1, the pusher member 70 may be an elongate member having a proximal end 72, and a distal end 74 sized for introduction into a body lumen and/or into the sheath 10. In addition, the pusher member 70 may include a lumen 76 that extends from a distal opening 78 to a side opening 77. In one embodiment, the lumen 76 may have a length of not more than about forty millimeters (40 mm) and/or between about ten to one hundred fifty millimeters (10-150 mm). Thus, the lumen 76 may be similar to a rapid exchange lumen, facilitating pushing a knot, pledget, or other component distally along a suture, as shown in FIG. 8G and explained further below.

The distal end 74 of the pusher member 70 may have a reduced profile, e.g., having a diameter between about one to six millimeters (1-6 mm), to facilitate advancement through a patient's vasculature and/or through the instrument lumen 16a of the sheath 10. The pusher member 70 may be sufficiently long to reach a target location from outside the patient's body. The pusher member 70 may be substantially flexible, yet have sufficient column strength to allow the distal end 74 to be advanced over a suture or other rail, as explained further below.

Turning to FIGS. 8A-8F, a method is shown for delivering a suture into a body lumen using an apparatus 8, which may be any of the apparatus described herein. As shown, the apparatus 8 may be used to deliver a suture 50 into a wall of a blood vessel or other body lumen. For example, turning to FIG. 8A, an aorta 90 is shown that includes an aneurysm 92 extending between the distal aorta 93 below the renal arteries 94 to the iliac arteries 95. A stent-graft 96 is shown that has been implanted within the aorta 90, e.g., extending across the aneurysm 92 into one or both iliac arteries 94, e.g., using known apparatus and methods.

The apparatus 8 may be used to deliver a suture 50 immediately after implanting the stent-graft 96, e.g., during an endovascular aneurysm repair (“EVAR”) procedure. Although not shown, it will be appreciated that one or more apparatus 8 may be used to deliver a plurality of sutures, e.g., around a periphery of the stent-graft 96. For example, a plurality of apparatus 8 may be provided in a kit, such that individual sutures may be delivered successively using the apparatus 8. Alternatively, a single apparatus 8 may be used to deliver a plurality of sutures (not shown), e.g., by reloading a suture and/or needle into the suturing catheter 30 and onto the suture carrier 40.

In another alternative, sutures delivered using one or more apparatus 8 may replace a stent or other anastamosis device on one or both ends of a tubular graft. For example, a fabric or other tubular graft may be delivered into the aorta, and the procedures described herein may be used to suture the ends of the tubular graft within the patient's vasculature. In a further alternative, the apparatus 8 may be used to deliver one or more sutures at a later time after completing an EVAR or other procedure, e.g., to seal an endoleak that has occurred, e.g., between a proximal end 97 of the stent-graft 96 and the wall of the distal aorta 93.

Turning to FIG. 8B, the distal end 14 of the delivery sheath 10 may be advanced into the aorta 90. In one embodiment, the delivery sheath 10 may be introduced through a percutaneous puncture (not shown), e.g., using an introducer sheath (also not shown) directed through the percutaneous puncture using known apparatus and methods. For example, the delivery sheath 10 may be introduced into a peripheral vessel, such as the femoral artery, carotid artery, radial artery, and the like, and advanced through the patient's vasculature into the aorta 90 and/or stent-graft 96. A guidewire 99 may be placed previously from the entry site into the aorta 90 using known methods, and the delivery sheath 10 may be directed over the guidewire 99 into the aorta 90 and/or stent-graft 96. For example, a proximal end (not shown) of the guidewire 99 (outside the patient) may by backloaded through the opening 17 in the distal end 14 and through the instrument lumen 16a (see, e.g., FIG. 2) or a dedicated guidewire lumen (not shown), allowing the delivery sheath 10 to be advanced over the guidewire 99.

Optionally, one or more dilators and/or obturators (not shown) may be used to facilitate delivery of the delivery sheath 10. For example, a dilator may be provided within the delivery sheath 10, e.g., within the instrument lumen 16a, having a tapered and/or other tip that extends beyond the distal end 14 of the delivery sheath 10. Such a dilator may provide a smooth and/or more gradual transition to the distal end 14, which may facilitate advancing the delivery sheath through tortuous anatomy. In addition, the dilator (or an obturator introduced through the instrument lumen 16a) may seal or close the window 20 to prevent tissue, blood, or other structures from entering and/or catching on the window 20.

With continued reference to FIG. 8B, the distal end 14 of the delivery sheath 10 may be positioned adjacent the proximal end 97 of the stent-graft 96 and/or rotated about the longitudinal axis 18, if necessary, such that the window 20 is oriented towards a target location where a suture is to be delivered. The balloon 22 may then be expanded until the distal end 14 and/or the window 20 are pressed against the wall of the aorta 90 and/or the stent-graft 96. For example, saline, air, nitrogen, or other fluid may be directed from a syringe or other source of inflation media (not shown) coupled to the proximal end 12 of the delivery sheath 10 (e.g., coupled to the side port 28b shown in FIG. 1). The inflation media may then be directed through the inflation lumen 16b (not shown, see, e.g., FIG. 2) and into the interior 23 of the balloon 22.

The balloon 22 may expand transversely away from the distal end 14, e.g., opposite from the window 20, until the balloon 22 frictionally engages and/or otherwise contacts the wall of the aorta 90 and/or the stent-graft 96. Thus, the distal end 14 and/or window 20 may be anchored or otherwise substantially stabilized relative to the surrounding structure(s), e.g., the wall of the aorta 90 and/or stent-graft 96. Optionally, the balloon 22 may be shaped or otherwise configured such that blood may continue to flow around the balloon 22 even after expansion. For example, the balloon 22 may have a relatively narrow width (transversely with respect to the longitudinal axis 18) such that the balloon 22 does not completely occlude the aorta 90, and blood may flow past the balloon 22. Any dilator and/or obturator within the delivery sheath 10 may be removed before or after anchoring the distal end 14 using the balloon 22.

Turning to FIG. 8C, with the distal end 14 of the delivery sheath 10 stabilized, the suturing catheter 30 may be introduced into the delivery sheath 10. For example, the distal end 34 of the suturing catheter 30 may be introduced into the port 28a (not shown, see FIG. 1) and advanced through the instrument lumen 16a until the suture carrier 40 is disposed adjacent the window 20. As explained above, the delivery sheath 10 and/or suturing catheter 30 may be configured such that the suture carrier 40 is automatically angularly aligned with the window 20. Alternatively, the suturing catheter 30 may be rotated within the delivery sheath 10, if necessary, to align the suture carrier 40 and window 20.

Optionally, the delivery sheath 10 and/or suturing catheter 30 may include one or more markers, e.g., radiopaque bands and the like (not shown), on the distal end(s) 14, 34. Thus, manipulation of the delivery sheath 10 and/or suturing catheter 30 may be monitored using fluoroscopy or other external imaging. For example, the markers may indicate when the suturing catheter 30 is properly positioned within the delivery sheath 10, e.g., when the suture carrier 40 is axially and/or angularly aligned with the window 20.

Turning to FIG. 8D (where the delivery sheath 10 has been omitted for clarity), with the suture carrier 40 oriented towards the window 20, the suture carrier 40 may be actuated to rotate in the first direction, i.e., direct the tip 40c transversely out of the suturing catheter 30, through the window 20, and into the adjacent structure. As shown, the suture carrier 40 includes a separate needle 60 having a sharpened point 60a that can penetrate through the wall of the stent-graft 96 and into the wall of the aorta 90. As the tip 40c of the suture carrier 40 travels along the curved path, the needle 60 may pass through at least a portion of the wall of the aorta 90 and stent-graft 96, reenter the aorta 90 and window 20, and pass through the noose 46a, thereby drawing the suture 50 through a tract 98 created by the needle 60, as shown. As shown, the tip 40c may initially travel substantially perpendicular to the longitudinal axis 18 of the suturing catheter 30, which may facilitate puncturing or otherwise driving the sharpened point 60a of the needle 60 (or of the suture carrier 140 itself, as shown in FIGS. 7A-7C) into and/or through the stent-graft 96 and/or wall of the aorta 90. In addition, this initial orientation of the tip 40c may maximize the “bite” or depth of penetration of the suture carrier 40, as explained above.

Turning to FIG. 8E, with additional reference to FIGS. 3B-3D, with the tip 40c and needle 60 extending through the noose 46a, the noose 46a may be tightened around the suture carrier 40. The suture carrier 40 may then be rotated in the second direction, i.e., retracted back along the curved path, leaving the needle 60 and/or suture 50 captured by the noose 46a, thereby stripping the needle 60 off of the tip 40c. Stated differently, the noose 46a may retain the needle 60 and suture 50, while the tip 40c is withdrawn from the needle 60 and the suture carrier 40 is returned to its original position, as shown in FIG. 8E. Alternatively, for example, as described above with reference to FIGS. 7A-7C, if the suture carrier 140 carries a suture 50 without needle 60, the suture 50 may be captured with the noose 46a and stripped off of the suture carrier 140 when the suture carrier 140 is retracted.

In another embodiment, the filament 46c may be pulled before retracting the suture carrier 140 to pull the needle 60 and/or suture 50 off of the tip 40c. In addition, if desired, the filament 46c may be pulled proximally to withdraw the needle 60 and/or suture 50 through the suturing catheter 30 into the proximal end 32, handle 48 (not shown, see FIG. 1), and/or completely out of the patient. Alternatively, the noose 46a may simply hold the suture 50 and/or needle 60 while the suturing catheter 30 is removed.

Turning to FIG. 8F, the suturing catheter 30 may be withdrawn from the aorta 90, leaving the suture 50 extending through the aorta wall and stent-graft. For example, if both ends 50a, 50b of the suture 50 are located outside the patient (with the suture 50 extending through the suture and capture device lumens 36a, 36b), the suturing catheter 30 may simply slide over the suture 50 as it is removed, and the suture 50 may remain substantially stationary. Alternatively, if one end 50a of the suture 50 is captured and held by the noose 46a, the suture 50 may move as the suturing catheter 30 is withdrawn. For example, if the one end 50a is withdrawn with the suturing catheter 30, the suture 50 may be pulled distally through the suture lumen 36a, slide through the tract 98, and into the aorta 90 trailing the distal end 34 of the suturing catheter 30.

Once the suturing catheter 30 is completely removed from the patient, both ends 50a, 50bof the suture 50 may extend from the entry site (not shown). If one or both ends 50a, 50b are coupled to the suturing catheter 30 (not shown), the end(s) 50a, 50b may be cut or otherwise severed. Thus, the result is a suture 50 having free ends 50a, 50b outside the patient that extends into the patient's vasculature and through the tract 98, as shown.

Turning to FIG. 8G, a knot 52 may then be advanced from the ends 50a, 50b of the suture 50 into the aorta 90, e.g., using knot pusher 70. For example, after tying a slipknot or other loop or knot 52 between the ends 50a, 50b of the suture 50, one end of the suture 50 may be backloaded through lumen 76 of the knot pusher 70 and out side opening 77. The knot pusher 70 may then be introduced into the percutaneous entry site and advanced into the aorta 90, thereby delivering the knot 52 against the wall of the aorta 90 and/or the stent-graft 96. As shown, the distal end 74 of the knot pusher 70 may be introduced into the instrument lumen 16a (e.g., via the port 28a in the handle 26, shown in FIG. 1) and advanced into the distal end 14 of the delivery sheath 10, which may be still stabilized at the target location by the balloon 22.

Alternatively, the delivery sheath 10 may be removed, and the knot pusher 70 may be advanced into the aorta 90 over the suture 50. Optionally, in this alternative, the knot pusher 70 may include a guidewire lumen (not shown) and the knot pusher 70 may be advanced over a guidewire into the aorta 90. For example, the guidewire may the same guidewire 99 used to introduce the delivery sheath 10, as described above. Alternatively, the suture 50 itself may guide the knot pusher 70 into the aorta 90. In yet another alternative, it may be possible to preload one or more knots (not shown) within the suturing catheter 30 that may be released and advanced to the target location.

Optionally, to tighten the knot 52, the distal end 74 of the knot pusher 70 may be directed at least slightly beyond the tract 98, whereupon the ends 50a, 50b of the suture 50 may be pulled. This action may tighten the knot 52 against the stent-graft 96 and/or wall of the aorta 90. If desired, this process may be repeated by removing the knot pusher 70 from the patient, forming another knot with the ends 50a, 50b of the suture 50, and advancing the knot pusher 70 over one end of the suture 50 to direct the knot into the aorta 90. It will be appreciated that a variety of knots may be formed in this manner, such as a square knot, a surgeon's knot, and the like.

Optionally, if desired, a pledget, e.g., small panel of felt or other material (not shown), may be advanced over the ends 50a, 50b of the suture 50 into the aorta 90 before tying the knot 52. For example, the pledget may include a pair of holes through which the ends 50a, 50b may be directed, whereupon the pledget may be advanced over the suture 50. For example, after loading the pledget on the suture 50, the knot pusher 70 may be advanced over one end of the suture 50 to push the pledget into the aorta and/or against the stent-graft 96 and/or the wall of the aorta 90. The pledget may distribute forces more evenly when the knot 52 is tied, e.g., to prevent tearing or otherwise damaging the stent-graft 96 and/or tissue of the wall of the aorta 90.

Alternatively, instead of knots, other devices may be used to secure the suture to the stent-graft 96 and/or the wall of the aorta 90. For example, a clip (not shown) may be advanced over the ends 50a, 50b of the suture 50 and into the aorta 90, similar to the pledget just described. Such a clip may slide freely in the distal direction, i.e., over the suture 50 into the aorta 90, but may include detents or other elements that prevent the clip from moving proximally along the suture 50. Thus, once the clip is directed over the suture 50 and against the stent-graft 96 and/or wall of the aorta 90, the clip may secure the suture 50.

Turning to FIG. 8H, once the suture 50 is adequately secured, the ends may be cut or otherwise severed. For example, a cutter (not shown) may be advanced through the delivery sheath 10 and/or over a guidewire into the aorta. The cutter may then cut both ends of the suture 50 immediately adjacent the knot 52 (or a clip or other device). The cutter may then be removed, along with the delivery sheath 10, guidewire, or other instruments (not shown), leaving the suture 50 and 52 within the aorta 90, as shown.

Optionally, as explained above, one or more additional sutures may be delivered into the aorta 90, e.g., adjacent to the suture 50 and knot 52. For example, the delivery sheath 10 may be used to deliver one or more additional sutures using the same suturing catheter (if reloaded with a new needle and/or suture, e.g., as described above), and/or using a new suturing catheter (preloaded with a needle and/or suture). For example, the balloon 22 may be deflated, and the delivery sheath 10 may be rotated about its longitudinal axis until the window 20 is offset by a predetermined angle relative to the first suture 50. This movement may be monitored, e.g., using external imaging, as explained above.

Once the delivery sheath 10 is properly oriented, the balloon 22 may be inflated again to anchor and/or stabilize the distal end 14 and window 20. A suturing catheter may be advanced into the delivery sheath 10 and used to deliver another suture, which may be tightened and/or severed, as described above. If an endoleak is being repaired, it may be necessary only to deliver a few, e.g., two or three, sutures. If a graft is being attached to a vessel wall, a plurality of sutures may be delivered completely around the periphery of the graft and/or vessel wall. Once sufficient sutures are delivered, the instruments may be removed, and the patient may recover similar to known procedures.

Although the methods just described involve delivering a suture through a stent-graft, it will be appreciated that one or more sutures may be delivered as described herein during other procedures and/or involving other locations within a patient's body. For example, a suture may be delivered at any location within a patient's vascular system, gastrointestinal system, and the like. It may also be possible to deliver sutures without using the delivery sheath described. For example, a suturing catheter may be introduced through other devices, such as an endoscope, colonoscope, and the like. Alternatively, a balloon or other anchoring device (not shown) may be provided on a suturing catheter 40 itself, e.g., opposite to slot 38 above the suture carrier 40.

In an exemplary procedure, one or more sutures may be delivered to a bleeding site, e.g., a duodenal or other ulcer, an arteriovenous malformation within the gastrointestinal tract or other body structure, to stop bleeding. Alternatively, one or more sutures may be delivered to close a fistula, e.g., pancreatic, gastric, and/or esophageal fistulas, or fistulas in communication with the small intestine, colon, or rectum. In another procedure, one or more sutures may be delivered around an annulus of a valve or through one or more leaflets of a valve, particularly at or near a commissure, e.g., to tighten a mitral, aortic or other valve within the heart. In yet another procedure, one or more sutures may be delivered to secure other prostheses within a patient's body in addition to stent-grafts, such as prosthetic heart valves.

In another exemplary procedure, one or more sutures may be delivered during a gastric bypass or other laparoscopic or endoscopic procedure. For example, one or more sutures may be delivered to secure an anastamosis, e.g., connecting the esophagus, stomach, colon, or other organs within which a portion has been removed and/or bypassed.

In yet another alternative, a suturing catheter may be provided that includes multiple suture carriers (not shown) rotatably mounted to the distal end. For example, a suturing catheter may include a pair of suture carriers that are offset from one another about one hundred eighty degrees (180°). Thus, a first suture carrier may be directed transversely out of one side of the suturing catheter while a second suture carrier may be directed transversely out of the opposite side of the suturing catheter. The suture carriers may be actuated independently of one another or simultaneously. Such a device may be useful for repairing a mitral valve (e.g., at the commissure) or for closing a Foramen Ovale (“PFO”).

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

We claim:

1. An apparatus for delivering a suture within a patient's body, comprising:
an elongate tubular member comprising a proximal end, a distal end sized for introduction into a body lumen, and a lumen extending between the proximal and distal ends; and
a suture carrier rotatably mounted to the distal end, the suture carrier comprising a tip for carrying a suture thereon, the suture carrier being rotatable in a first direction such that the tip travels outwardly from a side of the distal end along a curved path until the tip reenters the distal end, the suture carrier being rotatable in a second opposite direction for retracting the tip back into the distal end along the curved path.

2. The apparatus of claim 1, further comprising a capture device positioned on the distal end for capturing a suture carried on the tip after the suture carrier is rotated in the first direction.

3. The apparatus of claim 2, wherein the capture device comprises a noose on a distal end of a filament extending from the distal end towards the proximal end of the tubular member.

4. The apparatus of claim 3, wherein the filament may be withdrawn from the proximal end of the tubular member, causing the noose to pass proximally through the tubular member to the proximal end.

5. The apparatus of claim 1, further comprising an expandable anchoring member on one side of the distal end for directing an opposing side of the distal end against a wall of a body lumen within which the anchoring member is expanded;

6. The apparatus of claim 1, further comprising a needle carried on the tip, and a suture extending from the needle through the lumen of the tubular member to the proximal end.

7. The apparatus of claim 6, wherein the needle comprises a sharpened point extending from the tip.

8. The apparatus of claim 6, the needle further comprising a recess for receiving the tip of the suture carrier therein.

9. The apparatus of claim 1, further comprising a suture releasably carried by the suture carrier.

10. The apparatus of claim 9, wherein the tip of the suture carrier is sharpened for penetrating through tissue, the suture carrier further comprising a pocket for releasably secured the suture to the suture carrier.

11. The apparatus of claim 1, further comprising an elongate sheath comprising a proximal end, a distal end sized for introduction into a body lumen, and a lumen extending between the proximal and distal ends sized for receiving the tubular member distal end therein, the sheath comprising a window in a sidewall of the sheath distal end through which the suture carrier may pass when the suture carrier travels along the curved path.

12. The apparatus of claim 11, further comprising an expandable member on the sheath distal end generally opposite the window for directing the window against a wall of a body lumen within which the expandable member is expanded.

13. The apparatus of claim 11, wherein at least one of the tubular member distal end and the sheath lumen are keyed such that the tubular member distal end is received within the sheath distal end such that the suture carrier is aligned with the window.

14. The apparatus of claim 11, wherein the sheath lumen comprises a stop for preventing further distal movement of the tubular member into the sheath lumen when the suture carrier is disposed adjacent the window.

15. A method for delivering a suture at a remote location within a patient's body, comprising:
advancing a distal end of a tubular member into a body lumen;
directing a needle outwardly from the distal end along a curved path such that the needle penetrates a wall of the body lumen, passes through the wall, and reenters the body lumen, the needle being coupled to one end of a suture;
capturing the one end of the suture;
withdrawing the one end of the suture proximally from the body lumen; and
advancing a knot over the suture into the body lumen to secure the knot to the wall of the body lumen; and
severing the suture to leave the knot in the body lumen.

16. The method of claim 15, wherein the needle is carried on a suture carrier rotatably mounted to the tubular member distal end, the suture carrier being rotated in a first direction to direct the needle along the curved path.

17. The method of claim 16, further comprising rotating the suture carrier in a second direction opposite the first direction to withdraw the suture carrier back into the tubular member distal end.

18. The method of claim 15, wherein:
the needle at least partially enters the tubular member distal when the needle reenters the body lumen;
at least one of the needle and the one end of the suture is captured by a noose within the distal end; and
the noose is pulled proximally from the body lumen to withdraw the one end of suture from the body lumen.

19. The method of claim 18, wherein the suture is disengaged from the needle when the noose is pulled proximally from the body lumen.

20. The method of claim 15, further comprising advancing an elongate sheath into the body lumen until a window in the elongate sheath is oriented towards a target tissue structure, wherein the tubular member distal end is advanced into the body lumen through the sheath, and wherein the needle is directed outwardly through the window along the curved path.

21. The method of claim 20, further comprising expanding an expandable member within the body lumen to substantially secure the window adjacent the target tissue structure.

22. The method of claim 21, further comprising rotating the sheath about its longitudinal axis to orient the window towards a target tissue structure.

23. The method of claim 15, further comprising advancing a cutter into the body lumen adjacent the tubular member distal end, wherein the cutter cuts the suture to sever the suture.

24. The method of claim 15, wherein the body lumen comprises a blood vessel adjacent an aneurysm, and wherein the needle penetrates through a portion of an endoluminal prosthesis extending from the blood vessel through the aneurysm.

25. An apparatus for delivering a suture within a patient's body, comprising:
an elongate tubular member comprising a proximal end, a distal end sized for introduction into a body lumen, and a lumen extending between the proximal and distal ends, and
a suture carrier rotatably mounted to the distal end.

26. The apparatus of claim 25, wherein the suture carrier is rotatable around a rotation axis that extends transversely relative to a longitudinal axis of the tubular member.

27. The apparatus of claim 26, wherein the rotation axis is disposed adjacent a sidewall of the distal end to maximize a penetration distance of the suture carrier away from the sidewall.

28. The apparatus of claim 26, wherein the rotation axis is disposed not more than about two millimeters from the sidewall.
